(19) United States
(12) Patent Application Publication
(10) Pub. No.: US 2005/0130827 A1
(43) Pub. Date: Jun. 16, 2005

(54) Decomposable monotithic ceramic materials having an at least bimodal pore distribution and active metal centers located in the pores

(76) Inventors: Stephan Andreas Schunk, Heidelberg (DE); Dirk Demuth, Walldorf (DE); Mika Linden, Abo (FI); Jan Henrik Smatt, Abo (FI)

Correspondence Address: Stephen D Scanlon;Jones Day, North Point, 901 Lakeside Avenue, Cleveland, OH, 44114, US

(21) Appl. No.: 10/505,384

(22) PCT Filed: Feb. 22, 2003
(86) PCT No.: PCT/EP03/01750

(30) Foreign Application Priority Data
Feb. 22, 2002 (DE) 10207504.2
Mar. 15, 2002 (DE) 10211600.8

(51) Int. Cl. B01J 21/04; B01J 23/02; C04B 38/00; B01J 31/00

(52) U.S. Cl. 501/80; 502/439; 502/150

(57) ABSTRACT

The present invention relates to decomposable monolithic ceramic materials having an at least bimodal pore structure, in particular having micropores and mesopores or mesopores and macropores or micropores, mesopores and marcopores, optinally having metal centres located in the pores. The invention further relates to processes for producing the materials of the invention and to the use of the materials of the invention and the materials produced by one of the processes of the invention, in particular in catalysis and catalyst research and in medical technology and for the time-delayed release of active compounds in the pharmaceutical industry.

The present invention relates to decomposable monolithic ceramic materials having an at least bimodal pore structure, in particular having micropores and mesopores or mesopores and macropores or micropores, mesopores and marcopores, as well as to said materials having metal centers located in the pores. The invention further relates to processes for producing the materials of the invention and to the use of the materials of the invention and the materials produced by one of the processes of the invention, in particular in catalysis and catalyst research and in medical technology and for the time-delayed release of active compounds in the pharmaceutical industry.

In general, porous ceramic materials can be produced by a large number of processes; in the following presentation of the prior art, a distinction will be made between three groups of materials which are produced by correspondingly different processes: (i) materials having micropores and mesopores, (ii) materials having macropores and mesopores, and (iii) materials having micropores, mesopores and macropores. It should be stated right at the beginning that the porous materials produced by processes coming under (i) are merely in the form of powder and not a stable monolith and the porous materials obtained by the processes (ii) and (iii) are not decomposable.

Porous ceramic materials having micropores and mesopores, in particular those which are produced using amphiphilic substances in sol-gel processes, are described by way of example in the following review article: D. M. Dabbs and I. A. Aksay, Ann. Rev. Phys. Chem. 51 (2000) 601-622. Although these materials have well-defined micropores and mesopores, they are obtainable only as powders. It is explicitly stated in the publication that “a necessary prerequisite for the possible commercial use [of such mesostructures] is the formation of controlled shaped bodies and structures in the form of continuous thin films, fibres and monoliths”, with the size of these shapes being said to be “above the microscopic particle size which has hitherto been synthesized”.

An optically isotropic and transparent monolith can be obtained, at least temporarily, by use of a liquid crystal phase as template in a sol-gel process (cf., for example, K. M. McGrath et al., Langmuir 16 (2000, 398-406), but the monolith silicate structure disintegrates into a powder on drying. Analogously, the use of surface-active agents (surfactants) of the prior art, for example, ionic surface-active agents, also leads to structures which disintegrate on drying and/or calcination (cf., for example, C. H. Ko et al., Microporous and Mesoporous Materials 21 (1998) 235-243). Unsatisfactory dimensional stability, i.e. a pulver morphology, is even more pronounced in the case of the known MCM-41 materials which were developed for the first time in 1992 in research laboratories of the Mobil company.

In general, microporous and mesoporous structures can be produced and varied particularly advantageously when nonionic block copolymers are used in place of the conventional ionic surface-active agents in the sol-gel process. Particular mention may be made in this context of the work of G. D. Stucky and his group (cf., for example, D. Zhao et al., Science 279 (1998) 548-552). In these processes, amphiphilic triblock copolymers act as templates for pore formation and determine the porosity of the silicon framework. The framework is in this case built up in a sol-gel process by hydrolysis of a silicate precursor material. The mesostructures formed here display well-defined Bragg reflections in the X-ray powder diffraction pattern, in particular also in the small angle region. This indicates a high degree of order on a mesoscopic length scale which is, however, only achieved on calcination (cf., for example, P. Yang et al., Nature 396 (1998) 152-155). The important advantage of these processes is that block copolymers, in contrast to conventional surface-active agents, allow virtually continuous variation of the pore parameters, and this can be done in situ during the synthesis. It can be achieved, for example, by adjustment of the ratios of amounts, the composition, the molecular weight or the molecular architecture of the block co-polymers in the mixture. However, disadvantages are that all processes of the prior art in which these block copolymers are employed do not enable macropores to be obtained and, in particular, that this porous material, too, is pulverulent and not in the form of a monolith material.

Another class of sol-gel processes is based on the use of water-soluble polymers as templates. In contrast to the abovementioned processes, materials having mesopores and macropores but no micropores are obtained in this way. A significant advantage of these materials is that monolith shaped bodies and not just powders can now be obtained. In this context, particular mention may be made of the publications of Nakanishi and colleagues. Thus, for example, WO 95/03256 describes the production of porous monolithic materials having macropores together with mesopores. These materials are obtainable by hydrolysis of silicate precursor materials with subsequent sol-gel condensation in the presence of water-soluble polymers, for example poly(ethylene oxides) (PEO).

The significant features of the synthesis of porous monolithic ceramics by the method of Nakanishi are (i) the change of solvent between an acidic solvent and a basic solvent, which makes a significant contribution to enabling pore size and pore size distribution of the mesopores to be controlled in a targeted manner, and (ii) the calcination of the green body obtained after drying at high temperatures, i.e. at at least 600° C. This calcination step is essential to the solution of the object of the invention stated in WO 95/03256, namely the provision of vitreous columns which are stable to their environment and are fully hardened and are tailored, in particular, for applications in high-pressure liquid chromatography (HPLC). Silicate columns produced by this process are manufactured and marketed as Chromolith™ by Merck (Darmstadt, Germany) and by EM Science (Gibbstown, N.J., USA).

A disadvantage of the above-mentioned process described by Nakanishi is, in particular, the fact that the vitreous monolith obtained is dimensionally stable but is also inert toward physiological environmental conditions (see the definition given below), in particular is not soluble in water or aqueous solutions. This is disadvantageous for, in particular, delayed and time-controlled release of active compounds in pharmacy and for the design of biodegradable or resorbable bioceramics in medical technology. The processes of the prior art thus lead exclusively to porous ceramic structures which are not degradable in the human body or are not suitable for the release of active compounds. Furthermore, the lack of micropores in the monoliths produced by the Nakanishi method has to be regarded as a deficiency, since trimodal porous monoliths are not obtainable by this synthetic route.

In addition to porous materials that may be used, among others, as catalytically active materials, materials having catalytically active metal centers are described quite generally in the prior art. For example, mention may be made of the publications of the Fina company which relates to inter alia, solid, particulate catalyst systems based on metallocenes or metal salt solutions which are particularly useful for the polymerization and/or copolymerization of olefins (cf., for example EP 0 573 403, WO 98/02236 or U.S. Pat. No. 5,846,896). These documents demonstrate the general importance of active metal centers which can be obtained, for example, by immobilization of metallocenes on a support material. In the scientific literature, mention may also be made of the work of J.-M. Basset and colleagues (cf., for example, V. Vidal et al. Science 276 (1997) 99-102) in this context. However, the prior art gives no information as to how such active centers based on metallic compounds generally or on metallocenes can be obtained in combination with the decomposable monolithic material of the invention.

In general, the prior art does not describe any ceramic materials which are at the same time microporous, mesoporous and macroporous and, in particular, does not describe any materials which are, in addition, monolithic and decomposable under physiological conditions. In addition, the prior art does not disclose any process which would be suitable for producing such materials. Thus, EP 0 978 313 A1 does describe a material having a trimodal porosity (micropores, mesopores and macropores), but this is only obtainable as a composite comprising active carbon in a silicate framework, i.e. the trimodal porous structure is obtained in a subsequent step and not intrinsically and in situ during the production process. Furthermore, this material is not a ceramic material (see definition given below) for the purposes of the present invention, since active carbon as substantial constituent means that the material is not a ceramic material.

The prior art regarding the controlled and/or time-delayed release of active compounds is essentially characterized by conventional techniques, i.e. the pressing of tablets containing slowly dissolving components or the encapsulation of the active component in a shell which decomposes in the stomach, (cf., for example JP 632 430 36, in which the retention of active compounds by mixing silicates and cellulose is described, and also U.S. Pat. No. 5,869,102, which is similarly concerned with the joint pressing of active compound, colloidal silicates and microcrystalline cellulose). These are obviously processes in which the active compound is released in macroscopic amounts, i.e. in pulses, after decomposition of the shell and/or other constituents, the release rate is essentially independent of the active compound used and local application and the release rate cannot be varied over a particularly wide range or be controlled particularly precisely.

Accordingly, it is desirable and the objective of significant efforts in research and development to immobilize active compounds in microscopic or mesoscopic “cages” in a targeted and active compound-specific manner and then to release them at a predetermined point in time in a specific location (e.g. gastrointestinal tract, forming tissue in the healing of wounds, etc.) by dissolution of the cage structure. Such an approach is described, for example, in F. Caruso et al., Chem. Mater. 11 (1999) 3309-3314. This document is concerned with microvoids which are obtained by coating colloidal spherical templates with nanosize particles and polymers. Such hollow microstructures can, at least theoretically, be used for encapsulating active compounds, cosmetic substances or dyes and then releasing them in a targeted manner. However, these materials are merely clusters in solution and not a macroscopic monolithic material.

The general methodology of the targeted delivery of active protein and peptide compounds (PP drugs), in particular by the peroral route, i.e. by oral intake and subsequent entry into the bloodstream via the gastrointestinal tract, is described, for example, in A. Sood and R. Panchagnula, Chem. Rev. 101 (2001) 3275-3303. For the purposes of the present invention, the approach described in this document as an in-principle possibility, namely carrying out the resorption of PP drugs from the gastrointestinal tract into the bloodstream with the aid of nanosize particles, is of particular interest. It has been found that particles having a size of up to 5 μm can in actual fact be transported intact and without any change in their properties through the intestinal wall into the bloodstream.

Mention may finally be made of a recently published process for producing porous carbon-containing powders/crystallites using siliceous templates. In this process, carbohydrate-containing substances or carbohydrates as precursor compounds are brought into contact with a siliceous template, e.g. MCM-48 and MCM-41. After drying and calcination, the siliceous template can be removed, e.g. using HF and/or NaOH, to leave a carbon-containing porous powder or a porous powder consisting entirely of carbon, having the pore structure of the siliceous template. Particular mention may be made of the following two publications: M. Kruk et al., J. Phys. Chem. B 104 (2000) 7960 and R. Ryoo, S. H. Joo and S. J. Jun, J. Phys. Chem B 103 (1999) 7743. However, according to the prior art, monolithic shaped bodies cannot be obtained by this process.

It can therefore be stated in summary that although there are monolithic ceramic materials having micropores and mesopores and corresponding materials having mesopores and macropores, there are no monolithic ceramic materials which are dimensionally stable but nevertheless decompose under physiological conditions, i.e. in particular in contact with water. Such materials are of particular interest for the targeted and time-delayed release of active compounds and in medical technology.

Furthermore, the prior art discloses no monolithic ceramic materials which have pores at three levels, i.e. in principle the atomic level, the nanosize level and the micro level. Such hierarchical materials are of particular interest in catalyst research and nanotechnology.

Finally, there are no known ceramic materials which have at least bimodal pore structure with active metal centers and are at the same time monolithic and decomposable.

It is accordingly an object of the invention to provide a novel ceramic monolithic material which has micropores and mesopores or mesopores and macropores or micropores, mesopores and macropores and which should be decomposable under physiological conditions, i.e. in particular for use in the pharmaceutical industry, medical technology, the cosmetics industry and the food industry. The materials used therefore have to be, in particular, biocompatible, nontoxic and biodegradable. A further important feature for the mass production of such materials is that the base materials should be cheap and available in large quantities, i.e. the use of expensive block copolymers, for example should be avoided. Furthermore, it is an additional but optional object of the invention to modify the novel porous ceramic material in such a way that at least one active metal center is present within the pores. Part of the object is, in particular, to provide a possible way of varying the acidity and/or the redox behavior of the porous monolith of the invention.

The specific shape and/or size and/or porous structure of the monolith of the invention should be defined in the production process itself and not only in a subsequent shaping step, as would be the case, for example, in the compaction of a porous powder. The decomposable but dimensionally stable monolith should also be characterized by an amorphous structure, i.e. a structure having no grain boundaries between crystallites which could lead to crumbling of the material. Such an amorphous structure is indicated, for example, by the absence of Bragg reflections in the X-ray diffraction pattern.

A particular object of the invention is also to provide monolithic ceramic materials having mesopores, i.e. pores having a diameter in the range from 2 nm to 50 nm, in which the pore diameter can be set in a targeted manner and varied over a wide range. Pores of this diameter are naturally of particular importance for applications of nanotechnology.

The object of the invention is achieved by providing a dimensionally stable and coherent monolithic ceramic material which can be regarded essentially as a hardened but not completely cured material, i.e. as an at least partially water-soluble, agglomeration of nanosize particles. In this material, the nanosize particles form a coherent framework, preferably a siliceous framework. On a complementary level, the coherent nanosize particles define a continuous, i.e. channel-like, network of micropores, mesopores (=nanopores) or micropores, i.e. material-free voids.

Such a material can be obtained by various sol-gel processes which are essentially characterized in that at least one framework precursor material, at least one substance capable of hydrolysing the precursor material and at least one water-soluble polymer are combined. The further addition of an amphiphile is optional, but is the basis of a particularly important embodiment. The mixture obtained in this way is then subjected to at least one sol-gel transition, a solvent replacement step and a drying step. Depending on the embodiment, a calcination step following the drying step is mandatory, with the temperature of the calcination step having to be chosen so that the material sets but does not harden to form an inert, vectoreous body, i.e. the material in each case remains at least partially decomposable under physiological conditions.

The incorporation of active metallic centers which relates to another important optional embodiment can be achieved either (i) in the sol-gel process for the production of the material of the invention by (co)hydrolysis of decomposable precursor materials in which metals are present or else by (ii) bringing the green body according to the invention or the corresponding calcined materials into contact with readily hydrolysable precursor compounds in which metals are present, in particular metallocenes, in an after-treatment step. Both the acidity of the overall porous material and its redox behavior can then, as required by the object of the invention, be set at will via the type and amount of the active metal centers introduced by means of (i) and/or (ii). These two parameters can be set, in particular, independently of one another since the two methods (i) and (ii) of introducing active metal centers are based on different principles and thus influence the acidity and redox behavior in different ways.

Definitions essential for the understanding and interpretation of the present invention are given below.

A property which is of particular importance for characterizing the material of the invention is its morphology, i.e. the material should not be in the form of a powder but in the form of a coherent shaped body which, for the purposes of the present invention, is referred to as a monolith. For the purposes of the present invention, monoliths as coherent shaped bodies should have a dimension of at least 1 millimeter in all three directions in space. In principle, the monolith can have any shape, i.e. for example plates, rods, spears (beads, pellets) or any other conceivable geometric form. In particular, monoliths can also have complex shapes, for example, notches or grooves on the outsides with the aim of improving transport and/or flow properties around the monolith. In a practical embodiment, rods having a diameter of 5 mm and a length of from 5 to 10 mm are used.

For the purposes of the present invention, a ceramic material is any material which has a higher proportion (in percent by weight) of inorganic constituents than of organic constituents after the dry step. Organic constituents are, according to the invention, all constituents which contain exclusively compounds of carbon and optionally nitrogen, hydrogen, or oxygen. All other compounds are regarded as inorganic constituents, in particular also those constituents which are referred to as “organometallic” or “organosilicone” compounds in chemistry text books.

Porous material quite generally are characterized, inter alia, by their pore size, pore size distribution, type of pores simultaneously present, wall thickness of the frameworks surrounding the pores and their pore volume (porosity). On the basis of the pore size, a distinction is made between microporous materials, mesoporous materials and macroporous materials. The terms “microporous”, “mesoporous” and “macroporous” are used in the context of the present invention as they are defined in Pure Appl. Chem., 45 (1976), p. 79, namely as pores whose diameter is above 50 nm (macroporous) or in the range from 2 nm to 50 nm (mesoporous) or below 2 nm (microporous).

The pore size distribution can be narrow or broad, unimodal (one predominant diameter), bimodal (two pore types of different size are simultaneously present, with the mean pore sizes being further apart than the sum of the two widths at half height) or trimodal (three types of pores of different size are simultaneously present, with the mean pore sizes of adjacent types of pores being further apart than the sum of the corresponding two widths at half height). The pore size distribution (PSD) can be obtained, for example, by means of absorption measurements. In the case of the present invention, the pore size distribution can be approximated well by a Gaussian bell curve. The maximum of the bell curve gives the mean pore size. The full width at half height, i.e. the width of the function at half the height between baseline and maximum, is a measure of the broadness of the distribution. For most applications, a very narrow pore size distribution is sought.

Owing to inaccuracies in the methods of determining pore sizes, the assignment of pore types is undertaken within an error bar of two widths at half height. Thus, for example, measurements on one of the materials according to the present invention give a mean pore diameter which is located at about 1 μm (1000 nm) at a width at half height of half a micron. These pores are thus unambiguously micropores. At the same time, the same material contains a further type of pore having a mean pore diameter of 20 nm and a width at half height of 10 nm, i.e. these pores are unambiguously mesopores. Finally, the material contains a third type of pore whose diameter is determined as 3 nm with a width at half height of 1 nanometer and which, within the error range indicated above, can be assigned as micropores. In any case, this material is a material having a trimodal pore structure, since all three mean pore diameters are significantly further apart than the sum of the relevant widths at half height.

Porous materials having a multimodal, i.e. at least bimodal, pore size distribution are also referred to as hierarchical. Different pore sizes are typically associated with different transport behavior. Thus, for example, macropores are predestined for, in particular, “macroscopic” transport processes such as viscose or diffusive mass transport, while in the case of mesopores, interfacial diffusion and capillary effects dominate, and only activated transport takes place in micropores. Hierarchical pore structures thus result in hierarchical transport processes. It is thus possible, for example, for molecules to be localized in one place in micropores for a prolonged period, but then be rapidly transported away from this place by capillary or diffusive transport in mesopores or macropores on activation or dissolution of the micropores. Such a mechanism is, for example, of interest for the positionally and temporally localized release of active compounds in pharmaceutical applications. Similarly, a hierarchical pore structure is also of interest for catalytic systems in which the rapid transport of starting materials and products to and away from the catalytically active centers which are distributed in micropores having a high surface area occurs through mesoporous and macroporous transport channels.

For the purposes of the present invention, the term “decomposable” refers to dissolution and/or decomposition of the framework substance of the porous monolithic ceramic material under physiological conditions. Physiological conditions are all conditions which can occur in a living organism, in particular in the human body, especially in aqueous solution, in saline aqueous solution, in acidic aqueous solution, in alkali aqueous solution and also particularly in saliva, in bodily excretion and transportation, especially in sweat, and in all secretions from the body, in particular in mucus, and also in stomach juices, in the intestinal tract, in blood or blood plasma, in connective or muscle tissue and in bones or cartilage.

The novel monolithic ceramic material of the present invention is characterized in that it is at least bimodal and in that it is at least partially decomposable under physiological conditions, with the term “decomposable” being as defined above. In a preferred embodiment, the at least bimodal material contains micropores and mesopores or mesopores and macropores or micropores, mesopores and macropores, with the latter case being particularly preferred.

In a further preferred embodiment, the material of the invention is characterized in that it displays no Bragg reflections in X-ray measurements in the small angle region, i.e. in the angle range from 1° to 5° (when using a commercially available X-ray source, i.e. a conventional X-ray tube or a rotating anode which emits Cu—K_{α} radiation). Bragg reflections are the sharp peaks, i.e. peaks having a half width of a few widths at half height, known to those skilled in the art which indicate the presence of long- or intermediate-range order and can be employed for indexing 2- or 3-dimensional ordered structures. The characteristic Bragg reflections are found in the case of the microporous and mesoporous materials obtainable according to the prior art (cf. for example, D. Zhao et al., Science 279 (1998), page 549) but not in the case of the materials of the invention.

The material of the invention is, in a preferred embodiment, also characterized in that it has not been heated to above 500° C. at any stage during its pre-treatment, production and/or after-treatment. This ensures that the material is not cured completely to form a vitreous material or a glass ceramic which would then not undergo at least partial decomposition under physiological conditions. The fact that the material of the invention must not be heated to above 500° C. during its pre-treatment, production and/or after-treatment does not rule out exposure of the material of the invention to a temperature higher than 500° C. at some stage during its use, in particular in catalytic applications.

In a further embodiment, the monolithic ceramic porous material of the invention is characterized in that it has at least one further property selected from the following group, or has all of these properties:
the macropores are connected to form continuous transport channels within which mass transport from one part of the monolith to at least one further part can take place;
the size of the macropores, represented by the mean pore diameter of plus/minus an error limit of two full widths at half height, is from 50 nm to 1000 μm, preferably from 0.1 μm to 100 μm, particularly preferably from 0.5 μm to 30 μm;
the size of the mesopores, represented by the mean pore diameter plus/minus an error limit of two widths at half height, is from 5 nm to 50 nm, preferably from 10 nm to 40 nm;
the size of the micropores, represented by the mean pore diameter plus/minus an error limit of two widths at half height, is preferably from 0.5 nm to 4 nm, particularly preferably from 1 nm to 3 nm;
the size distribution of the mesopores is such that the full width at half height of the pore size distribution function is ideally not more than 100% of the mean pore width, particularly preferably not more than 50% of the mean pore width;
the material according to the invention after the drying step has a higher proportion, measured in percent by weight, of inorganic constituents than of organic constituents;
the material according to the invention after the drying step comprises silicon and oxygen as main constituents, measured in percent by weight.

The decomposable monolithic ceramic material of the invention having an at least bimodal pore structure can be produced by any conceivable method which leads to the above-mentioned material. In a preferred embodiment, it is produced by a process comprising at least the following steps:
(I) bringing a precursor material, a water-soluble polymer and a hydrolysis catalyst into contact with one another;
(II) inducing the sol-gel transition of the mixture from (I);
(III) removing and replacing the solvent in the gel from (II) or removing or replacing the solvent;
(IV) drying the green body obtained from (III);
(V) calcining the dried green body at temperatures which do not exceed 500° C. at any stage.

This preferred embodiment is a sol-gel process, i.e. polymerization of a molecular precursor material typically characterized by hydrolysis of the precursor material with subsequent condensation, i.e. formation of an oxidic network. Depending on condensation conditions, in particular depending on pH, linear polymers and long gel times or dense clusters or nanosize particles and short gel times, as well as, naturally, all intermediate states, can be obtained.

The materials obtainable according to the invention are (i) decomposable under physiological conditions, (ii) biodegradable, (iii) nontoxic and generally compatible with living organisms and allow, at least in one embodiment, (iv) setting of a trimodal pore structure which has not hitherto been known for these materials. Any combination of (i) and (iv) is conceivable.

The minimum starting materials to be used in the present embodiment are: (i) a precursor material, (ii) a substance suitable for hydrolysing the above-mentioned precursor material, hereinafter referred to as hydrolysis catalyst, and (iii) a water-soluble polymer.

Hydrolysis and condensation of the precursor material results in the framework of the monolith to be produced in the sol-gel process. As precursor materials, it is in principle possible to use all polymerizable substances having a low molecular weight, in particular: metal alkoxides, metal alkoxides having at least one nonhydrolysable group; polymerizable metal salts, in particular metal halogenides, and metal hydroxides such as aluminium, iron, or bismuth hydroxides, and also coordination compounds containing carboxyl or β-dicetone ligands. For the purposes of the present invention “metals” are not only the metallic conductors generally defined in textbooks, but also include semimetals and semiconductors, i.e., in particular, B, Si, Ge, As, Se, Sb and Te. Alkoxides include, in particular, tetraethoxysilane (TEOS), tetramethoxysilane (TMOS), tetrapropoxysilane (TPOS) and polymerized derivatives thereof; halides include halides which are decomposable in aqueous solution, in particular, chlorides such as SiCl₄, AlCl₄, TiCl₄, ZrCl₄, NbCl₅, TaCl₅, WCl₆ or SnCl₄. It is in principle also conceivable to use oligomeric precursor materials or organically modified silicates. A combination of two or more of the above substances is also possible.

The use of TEOS as precursor material is particularly preferred for the purposes of the present invention. In case active material centers are to be incorporated into the inventive porous materials, preference is given to the use of precursor materials containing metals in combination with siliceous precursor materials, in particular TEOS. As precursor materials containing metals, it is possible to use all metal-containing materials or metal-containing components or compounds mentioned in the preceding paragraph, with the hydrolysable alkoxides of Zr, Ti, Nb, Ta, Al, Sn and Pb being particularly preferred. Production of the monolithic and decomposable ceramics of the invention using only precursor materials containing metals is also conceivable.

The use of hydrolysable metal-containing components is a possible way of obtaining the material according to the invention having at least one active metallic centre integrated into the pore structure. A further possible route is indicated in the discussion of the after-treatment. For the purposes of the present invention, an “active metallic centre” is any metallic or metal-containing aggregation which comprises at least one metal atom and is a constituent of the internal and/or external surface of the porous material of the invention, i.e. can have a catalytic action.

A significant advantage of this method of producing an at least bimodal, porous and monolithic material is the opportunity of setting the acidity of the catalytically active metal centers in a targeted way. The metal can act as redox centre in a more or less strongly basic or acidic environment, for example determined by the ratio of TEOS to metal alkoxide and/or the use of admixed metal oxides or mixtures of metal oxides which are available in a wide variety and whose concentration can be varied over a wide range. The catalytically active material of the invention having metal centers of controlled acidity/basicity is accordingly superior to, for example, conventional catalysts for oxidational reactions (for example vanadium oxide on a support), since the inherent acidity of the latter catalysts leads to reduced selectivity and a sufficiently large amount of basic additives therefore has to be added subsequently.

As substance which leads to the hydrolysis of the precursor material, i.e. as hydrolysis catalyst, it is possible to use any substance which at least partially promotes the hydrolysis of the precursor materials. These can be basic or acidic substances, with acidic substances being preferred for the purposes of the present invention since they generally lead to formation of condensed clusters (nanosize particles). Basic substances worthy of mention are a ammonium, amines and ammonium hydroxide solutions. As acidic substances, mention may be of mineral acids such as nitric acid, sulphuric acid or hydrochloric acid and organic acids such as acidic acid and also, in particular, hydrofluoric acid (HF) and fluoride-containing solutions in general.

The use of dilute mineral acids, in particular dilute nitric acid, and dilute organic acids, in particular dilute acidic acid, as hydrolysis catalysts is particularly preferred for the purposes of the present invention.

Water-soluble polymers which meet the above-described requirements can be selected from the group consisting of uncharged polymers, in particular poly(ethylene oxides), poly(vinylpyrrolidones), poly(acrylamides), polyoles such as poly(ethylene glycols), polyoles with formamide; ionic polymers, in particular polyacrylate acids, poly(alkalimetal styrenesulfonates), poly(allylamines), and also combinations of two or more of the above-mentioned substances.

The use of polyethylene glycol (PEG) as water-soluble polymer is particularly preferred for the purposes of the present invention.

The medium in which the above-mentioned substances are combined in order to carry out a sol-gel process is typically an aqueous medium, which in the case of an acid being used as hydrolysis catalyst is naturally acidic. A media containing alcohol's or other solvents and/or or salts are likewise conceivable.

In addition to the above-mentioned substances which have to be combined as a minimum in order to obtain the material according to the invention in the sol-gel process of the invention, i.e. in addition to precursor material, hydrolysis catalyst and water-soluble polymer, it is possible to add, if desired, any further auxiliaries, active compounds or additives as long as these do not adversely affect the sol-gel process so that it can no longer be carried out as a whole. Such additional, optional materials can be selected from the group consisting of: salts, buffers, fibres, in particular cellulose; fillers; auxiliaries, active compounds, additives, fragrances or flavours immobilized in the framework, in particular pharmaceutical active compounds, enzymes, proteins, peptides; swelling agents; thickeners; dyes, pigments; metal-containing components, in particular metal ions or colloidal metals; polymerization initiators or inhibitors; and also combinations of at least two of the above-mentioned substances.

The actual process for producing the material according to the invention comprises bringing the above-described components, which in this composition are referred to as sol into contact with one another while stirring in a step (I) and introducing the mixture into a mould, with the mould naturally determining the exterior contours of the monolith. Specific ratios of amounts present in the sol and also absolute figures for weights are given in the examples. The range within which the ratios may vary in the preferred embodiments is indicated below. The specification of such amounts does not imply that other embodiments with different ratios are excluded from the scope of the invention, but instead serves merely for the purposes of illustration. These preferred ratios lead to a material which is, in the sense of the present invention, dimensionally stable, i.e. give a monolith, and also has at least some interlinked channels made up of connected macroscopic pores. The preferred molecular weight of PEG has been indicated above.

When TEOS and nitric acid are used together with PEG, a PEG content in the range from 2 to 10% by weight based on the total weight is preferred, with from 3 to 6% by weight being particularly preferred. In the case of a PEG content below 3%, the macropores are mostly isolated, while at a PEG content above 6%, the porous structure begins to break down and individual particles can be formed, i.e. the monolithic property is lost. The diameter of the macropores can be varied in the range from 1 to 80 μm by variation of the PEG content in the range from 3 to 6% by weight, with a high PEG content corresponding to a small diameter of the macropores.

The relative proportion of TEOS (again when using TEOS, PEG and nitric acid) is indicated by the “r value”, which is the molar ratio of water to Si (from the TEOS). For complete hydrolysis, for which four water molecules are naturally necessary, the r value is 4. In a preferred embodiment, the r value (ratio of water content to TEOS) is from 10 to 20, with an r value in the range from 12 to 18 being particularly preferred. At large r values, i.e. at a low Si concentration, particulate aggregates are formed, while at low r values the macropores are isolated, i.e. the transport channels are lost. The size of the macropores is likewise influenced by the r value, albeit not as greatly as by variation of the PEG content, and is in the range from 80 μm (small r value) to 5 μm (large r value).

It should be stated at this point that the size and interconnectivity of the macropores is determined essentially by the ratios of PEG to the total mass and of TEOS to water, while the size of the mesopores is determined essentially by the characteristics of the solvent replacement discussed below.

After the components have been combined in the above-mentioned ratios and the resulting sol has been introduced into the mould, the sol is converted into a gel at temperatures in the range from 20° C. to 80° C., particularly preferably about 40° C. When TEOS, PEG and nitric acid are used in the above-mentioned ratios, the gel time t_{gel} at 40° C. is from 4 to 6 hours (the optionally use of an amphiphile such as the CTAB, e.g. C₁₆ TAB, described below can result in a gel time in the range from 4 hours to 12 hours).

After this sol-gel transition, the gel is aged for at least 48 hours. Ageing can be carried out in temperatures in the range from 20° C. to 80° C., with a temperature of 40° C. being particularly preferred for the purposes of the present invention. Ageing in the sense of the invention is regarded as part of the sol-gel transition, i.e. part of step (II) and serves to strengthen the siliceous network (when TEOS is used) and thus to reduce or completely prevent crack formation in the monolith.

A next step in the process of the invention is step (III), namely the removal of the solvent or the replacement of the solvent. Solvent can be removed from the pores during ageing and drying. Solvent replacement is carried out by dipping the gel into an aqueous solution having an acidic or basic character, depending on whether an acidic solvent (here, for example, nitric acid) is to be replaced by a basic solvent or vice versa. Solvent replacement is essential to achieve a narrow pore distribution of the mesopores as a result of dissolution and redeposition of the matrix.

In the case of a gel which has been obtained from TEOS, PEG and nitric acid, replacement of the solvent by ammonium hydroxide solution is a preferred embodiment. The lower the concentration of ammonium hydroxide, the smaller the mesopores. The size of the mesopores can thus be adjusted by varying the ammonium hydroxide concentration, with concentrations of ammonium hydroxide of from 0.01 mol/l to 2 mol/l being preferred. The mean pore diameter of the mesopores is from about 3 nm in the case of a 0.02 molar solution to 16 nm in the case of a 2 molar solution. The values for the pore diameter were in each case obtained by means of Hg porosimetry.

For this step, preference is given to a temperature in the range from 40° C. to 95° C., particularly preferably from 70° C. to 90° C. The lower the temperature, the smaller the mesopores. The solvent replacement is typically carried out for from 5 to 10 hours, with lower temperatures requiring longer times. The volume of the replacement liquid should be about 10 times the volume of the monolith, but any value for the volume ratio is possible in principle as long as the monolith is immersed completely in the solvent. On the subject of the solvent replacement, the results of WO 95/03256 and of N. Ishizuka et al., J. of Chromatography 797 (1998) 133-137, are fully incorporated by reference into the present patent application.

Subsequent to the solvent replacement, the monoliths are washed (e.g. with 0.1 M nitric acid and subsequently with ethanol) and dried for 3 days at about 60° C. This washing and drying step is regarded, for the purposes of the present invention, as drying step (IV). The shaped body or monolith obtained in this way is referred as “green body” for the purposes of the invention and can be employed for the use provided for according to the invention. In particular, the green body can be subjected to any after-treatments, e.g. impregnation or bringing into contact with catalytically active substances, (pharmaceutical) active compounds, vitamins, enzymes, peptides, plant extracts, phytotherapeutic agents, fragrances and flavours, auxiliaries, nutrients, cosmetic substances, dyes, etc. The green body according to the invention is decomposable under physiological conditions, i.e. in particular, in contact with water. This means that all the above-mentioned substances can be liberated again at a future point in time at a predetermined location.

A preferred form of the after-treatment is functionalization of at least part of the internal surface formed by the micropores, mesopores or macropores or the channels resulting there from. It can, for example, be achieved by reaction of the free hydroxyl groups of the framework substance to convert them into different groups, for example ester groups. In a particularly preferred embodiment, for example, the internal surface which is hydrophilic because of the hydroxy groups can be made at least partially lipophilic.

In a further preferred embodiment of the after-treatment, a (preferably readily) hydrolysable precursor material containing at least one metallic component is brought into contact with the material according to the invention (a green body prior to calcination or shaped body after calcination). These can be brought into contact with one another under all conditions mentioned below in connection with controlled atmospheres, i.e., in particular, under reduced pressure or under a pressure above ambient pressure. They can be brought into contact by means of impregnation or deposition, with deposition being able to occur from the gas phase and/or the liquid phase. They can be brought into contact either continuously, consecutively, (i.e. the micropores first, then the mesopores and/or macropores) or in any desired steps and/or intermediate states. After the materials have been brought into contact with one another, hydrolysis of at least part of the hydrolysable material is induced. As the metal-containing precursor material, it is possible to use all metal-containing substances which bind to the OH groups present in the pores of the material according to the invention and/or to other conceivable functional groups to form ester groups or form a stable bond in another way. For the purposes of the invention, a bond is stable if it remains intact under the conditions of use. The metal-containing precursor materials can be selected from the group of metal-containing precursor materials mentioned above in general terms in connection with precursor materials. Organometallic compounds, metal salts and colloidal metals are preferred and metallocenes are particularly preferred.

The active metal centers formed in this way can, depending on the concentration of the metal-containing precursor material brought into contact with the interior walls of the pores, and depending on the functionality of the pores (density of the OH groups, reactivity of the OH groups, wetability of the pores, etc.), in principle occur in three different functional modifications: (a) as individual active centers (single site catalyst), (b) as nanosize particles bound to the surface of a pore and (c) in the case of a sufficient density of centers, as a film layer. Intermediate states, e.g. linear chains, branched paths or any combinations of the above-mentioned modifications are included as well.

What has been said with regard to the functional modification also applies analogously to active centers formed as described above by (co)hydrolysis of metal-containing precursor materials.

In a further preferred embodiment, the at least bimodal siliceous monolith of the invention is brought into contact with at least one carbon-containing precursor compound in the course of an after-treatment step. The carbon-containing compound can in principle be any substance which contains at least one carbon atom. Preference is given to alcohols and carbohydrates. For the purposes of the present invention, “bringing into contact” encompasses any introduction and/or application of the carbon-containing precursor compound into/onto the porous siliceous material. Preference is given to impregnation with a liquid solution, e.g. of sucrose or furfuryl alcohol. In the context of the present invention, the at least bimodal siliceous material according to the invention then acts as template.

After impregnation, the now carbon-containing material according to the invention can be treated further as described, for example, in the two publications M. Kruk et al., J. Phys. Chem. B 104 (2000) 7960 and R. Ryoo, S. H. Joo and S. J. Jun, J. Phys. Chem. B 103 (1999) 7743. In this context, both these publications are fully incorporated by reference into the present patent application. The further treatment is essentially a calcination at temperatures above 500° C., preferably at about 1000° C., under nitrogen and subsequent carbonization under reduced pressure. The siliceous template can then be removed if appropriate, for example, by treating the shaped body with HF and/or NaOH. This leaves an essentially carbon-containing porous monolith shaped body, or a porous monolith body consisting entirely of carbon, which has similar properties in terms of the pore structure as the siliceous shaped body used as template. An advantage over the prior art is that use of the monolith according to the invention as template also gives a carbon-containing monolith, i.e. not merely a porous powder.

In the calcination, i.e. in step (V), the green bodies are fired in an open or controlled atmosphere. The temperature can be in the range from the drying temperature to 500° C. Temperatures in the range from the drying temperature to 300° C. are particularly preferred. The calcination times depend on the desired degree of hardening and can range from a few hours to a few days. The main purpose of the calcination is, apart from the further hardening, to remove any undesirable organic components. In principle, calcination can be carried out before or after the after-treatment. Calcination prior to the after-treatment influences, inter alia, the number of OH functions and thus may also influence the after-treatment. For example, a reduced number of OH functions leads to a reduced number of metal-containing active centers.

For the purposes of the present invention, controlled atmospheres are: inert gases, reducing atmospheres, for example hydrogen-containing gases, hydrothermal conditions, in particular stream, oxidising atmospheres, reactive gases, atmospheres under superatmospheric or subatmospheric pressure and also all possible combinations and/or mixtures of the above-mentioned atmospheres.

In a further, particularly preferred embodiment, not only a monolithic ceramic decomposable material having a bimodal pore structure, i.e. with macropores and mesopores or with micropores and mesopores, but a monolithic ceramic decomposable material having a trimodal pore structure, in particular with macropores, mesopores and micropores, is obtained. Here, all process steps are analogous to the above-described steps (I) to (IV), with the exception that a fourth component, namely an amphiphilic substance, is added in addition to the precursor material, the hydrolysis catalyst and the water-soluble polymer in step (I):
(I) bringing a precursor material, a water-soluble polymer, and amphiphilic substance and a hydrolysis catalyst into contact with one another;
(II) inducing the sol-gel transition of the mixture from (I);
(III) removing and replacing the solvent in the gel from (ii) or removing or replacing the solvent:
(IV) drying the green body obtained from (III).

The amphiphilic substance has the task of providing a further template for pore formation (in addition to the water-soluble polymer which is responsible for phase separation). Owing to the nature of this template, it forms pores having a smaller diameter than that of the mesopores which are formed as a result of the addition of the water-soluble polymers. In particular, micropores, i.e. pores having a diameter of a few nano meters, are obtainable in this way. It needs to be particularly emphasized that in the process of the invention, the micropores formed during production of the material of the invention and are not introduced only in a subsequent after-processing step. The approach provided by the invention thus ensures that the distribution of the pores in the material is homogeneous. The amphiphilic substance is selected from the group consisting of block copolymers, in particular poly(alkyl oxide), triblock copolymers; surface-active agents (surfactants), detergents and soaps, in particular non-ionic alkyl poly(ethylene oxides); lipids, phospholipids; and also combinations of two or more of the above-mentioned substances.

For the purposes of the present invention, the use of ionic surfactants, in particular hexadecyltrimethylammonium bromide, as amphiphilic substance is particularly preferred. For the purposes of the present invention, all amphiphilic substances having a trimethylammonium bromide (TAB) unit and a carbon chain of any length are referred to as “CTAB”. The number of carbon atoms in an individual case may be specified, for example, C₁₆TAB in the present case. When an amphiphilic substance is used, calcination at temperatures above 500° C. may be necessary if complete removal of the organic amphiphile is desired. If TEOS, PEG, nitric acid and CTAB are used as starting materials, a CTAB content of from 0.01 to 5% by weight is preferred and a content of from 0.1 to 3% by weight is particularly preferred. Increasing the concentration of CTAB within the range indicated influences the mesopores, namely by decreasing their size, and also results in increased formation of micropores whose size extends from 1 nm to 5 nm and whose number increases with increasing CTAB concentrations.

As far as the incorporation of active metal centers and the steps of the after-treatment are concerned, what has been said above with regard to the corresponding bimodal material applies to the trimodal porous material of the invention as well.

As regards the production of carbon-containing porous monolithic shaped bodies or porous monolithic shaped bodies consisting entirely of carbon as a consequence of an after-treatment of the trimodal porous siliceous monolithic of the invention, what has been said above in the context of the bimodal monolith of the invention applies in full.

The monolithic ceramic material of the invention can be used advantageously in catalyst applications. This applies particularly to materials according to the invention which have active metal centers. As described above, these active metal centers can have been formed either in the sol-gel process or else by means of an after-treatment. Owing to the opportunity of controlling acidity and/or redox behavior of the active metal centers, the use of the catalysts of the invention for (partial) oxidation reactions is particularly preferred. It is naturally also conceivable for the material according to the invention to be catalytically active itself, i.e. without the presence of active metals, in particular as a result of its hierarchical pore structure. All materials described in the present invention can be used directly as catalysts, as support materials for catalysts and in catalyst research.

In a preferred embodiment, the acidity and the redox potential or the acidity or the redox potential of the metal-containing component(s) in a catalyst according to the invention containing a metal-containing component can be varied together or independently of one another by means of at least one measure selected from the group consisting of: concentration of the metal-containing precursor material relative to the precursor material containing no metal, type and composition of the metal-containing precursor material and density of the OH groups in the pores.

In related applications which exploit the pore structure, the material of the invention can be used as molecular sieve, ion exchanger or as biological separator having a sharp cut-off criteria in respect of the molecular weight, and also as osmotic membrane. Uses as waveguides, or support material for optical sensors; chemical or biological sensors are likewise conceivable. In all cases, and in the applications given below, the presence of active metals centers is optional. In some cases, it may be preferred to not use metal centers at all.

The above-described use of the material of the invention for the temporally delayed and temporally controlled and regionally defined supply of any substances, in particular dyes, cosmetic active compounds, auxiliaries or additives, nutrients or nutrient additives, animal feeds or animal feed additives, fragrances and flavours, is of particular importance. Particular preference is given to the delayed or temporally controlled, but in any case regionally well-defined, supply of pharmaceutically relevant active compounds in living organisms, in particular in the human body, is particularly preferred.

In this context, the hierarchical pore structure is of particular interest, since it makes it possible for the material of the invention to be successively loaded and unloaded. Thus, for example, the active compounds, auxiliaries or additives which are smallest can firstly be introduced into the micropores, followed by larger active compounds, auxiliaries or additives into the mesopores and/or macropores. When mixtures are incorporated, in particular plant extracts or phytotherapeutic agents, it is even possible to obtain a “natural” separation of various components in this way. It is also conceivable for cell constituents or relatively larger peptide chains or the like to be incorporated in the macropores in addition to the active compounds, auxiliaries or additives which are typically molecular in nature and can be incorporated in micropores or mesopores. In this context, attention may once again be drawn to the possibility of completely or partially functionalising the internal surface, in particular making it lipophilic. It is also conceivable for after-treatment and loading steps to be combined, i.e., for example, firstly introducing a hydrophilic substance into the micropores and subsequently making the remainder of the internal surface lipophilic and then introducing a lipophilic substance.

As noted above in the discussion of the prior art, a significant new development is to encapsulate active compounds, in particular active PP compounds (proteins and peptides) which are susceptible to enzymatic decomposition and cannot be resorbed per se through membranes, in nanosize particles and micro particles and thus, for example, transport them through the intestinal wall into the bloodstream. The material of the invention is particularly useful for such applications, since it can be regarded as an agglomeration of nanosize particles, with the cohesion of the nanosize particles being able to be adjusted by means of a variety of process parameters, for example, PEG or TEOS ? or degree of hardening, drying, calcination. The degree of agglomeration can be chosen so that the monolith remains intact until it enters the issue of the intestine and subsequently disintegrates into the nanosize particles. This naturally results in loss of the transport channels, but the active compound can still be retained in the mesopores or micropores of the particles and thus be carried in these fragments into the bloodstream. There, the nanosize particles can dissolve and liberate the active compound (without ever coming into contact with chemical or physical barriers).

In this context, it is of particular interest that the PEG used in the material of the invention is as such already known as a carrier for proteins, namely in that proteins are modified with PEG to increase their transportability (“pegnology”). This would, for example, appear to make it possible to incorporate proteins homogeneously into the material of the invention as early as in step (I) by using a mixture of PEG and PEG-modified protein in place of pure PEG. The material can then be solidified in the sol-gel process according to the invention and the monolith can be used directly as peroral formulation.

Preference is also given to the use of the material of the invention as biodegradable or resorbable (i.e. not calcined or hardened in another way) material and its use as biologically integrable ceramic material (bioactive, i.e. calcined, also known as bioglass) in medical technology, in particular for strengthening bone, for supporting connective tissue and for the healing of wounds.

As regards the above-mentioned carbon-containing monoliths formed by means of an after-treatment, preference is given, in addition to the applications mentioned above, to the following applications:—(i) use as resorbent for substances, e.g. in the case of poisoning phenomena in the human body, (ii) as hydrogen store, both as storage material as such and as support material for another storage material.

Carbon-containing monoliths which are based on the siliceous material of the invention as template and are formed, as mentioned above, in the after-treatment of the siliceous material of the invention are particularly useful as hydrogen stores or as supports for hydrogen stores since they have a large surface area due, in particular, to the hierarchical pore structure. In this context, the use of these carbon-containing monoliths in conjunction with finely divided hydrides is of particular interest. Here, particular preference is given to the hydrides of the main group metals and transition metals, e.g. magnesium hydride which is known as reversible hydrogen store. It is also conceivable for hydrides of semi-metals and non-metals to be used in combination with the carbon-containing monolithic material, with mixed hydrides, i.e. hydrides of main group and transition metals with semi-metals, being particularly preferred. Among mixed hydrides for the hydrogen stores, alanates which contain, for example, Li, Al and hydrogen and are typically used in combination with carbon-containing materials, for example carbon nanotubes, are of particular interest. In the context of the present invention, the alanates are used in combination with the carbon-containing hierherical monolithic material of the invention having an at least bimodal pore structure. A hydrogen store in which the hydrogen can be stored reversibly and released again is particularly preferred here.

Finally, it is also conceivable for the materials of the invention (with or without after-treatment) to be deposited as thin films or be used as building blocks in electric/electronic circuits. Here, their use as dielectrics having a high dielectric constant is of particular interest, since a large pore volume leads to correspondingly good electrical insulation, a property which is particularly important for the miniaturization of circuits in which “jumping over” of electric charge always presents a problem. Production and use of the materials according to the invention will be illustrated below with the aid of examples, without the generality of the claims according to the present invention being restricted thereby.

### BRIEF DESCRIPTION OF THE FIGURES

FIG. 1 shows a scanning electron micrograph of the inventive material obtained according to Example 3,

FIG. 2 shows the pore size distribution of decomposable inventive monoliths according to Example 5,

FIG. 3 shows the pore size distribution for micropores for decomposable inventive monoliths containing amphiphiles according to Example 7,

FIG. 4 shows the overall pore size distribution for the material of Example 7,

FIG. 5 shows the small angle x-ray diffraction pattern of the material of Example 7,

FIG. 6 shows the amount of a model drug set free by different embodiments of the inventive material according to example 10,

FIG. 7 shows the change in concentration of a model drug in a model bodily fluid as a function of time for the inventive decomposable material as described in Example 11.

### EXAMPLE 1

### Production of a Decomposable Monolith Having a Bimodal Pore Structure

In this example, the general synthetic route for producing a decomposable monolith having a bimodal pore structure will be described. Limits within which the proportion of individual constituents can be varied will be described in subsequent examples.

In the first step, 0.7 g of polyethylene glycol (PEG) having a molecular weight of 35000 is dissolved in 8.0 g of water and 0.81 g of 60% strength nitric acid. 6.5 g of TEOS are added to this mixture while stirring. The sol is stirred until a clear solution is obtained and is subsequently poured into moulds and gelled at about 40° C. for 5 hours and then aged at the same temperature for at least 48 hours.

To increase the degree of condensation and to control the size of the mesopores, the gel is subjected to solvent replacement at 90° C. in 1 M ammonium hydroxide solution for 9 hours. The volume of the solvent is about 10 times the volume of the gel. After solvent replacement is complete, the monoliths are washed with 0.1 M HNO₃ solution and 25% strength ethanol.

In the drying step, the green bodies are dried at 60° C. for 3 days. The monolith obtained after this displays all characteristic properties of the material according to the invention, for example, a bimodal pore structure, transport channels, absence of Bragg reflections. After the drying step, the monolith can be calcined at 450° C. for 5 hours, with the temperature being ramped up at 1 K/min.

### EXAMPLE 2

### Dependence of the Pore Structure on the Molecular Weight of PEG

The table at the bottom of this example shows how the pore structure (determined visually from scanning electron micrographs and by measurement of the porosity using Hg porosimetry) changes as a function of the molecular weight (MG) of PEG:

As can be seen from the table, the size of the macropores and the degree of mutual crosslinking can be adjusted by varying the molecular weight of PEG.

| | | | Pore type |
|---|---|---|---|
| MW of PEG | Transparency | Pore size (SEM) | (SEM) |
| 600 | transparent | none | none |
| 4 600 | transparent | <<1 μm | isolated |
| 10 000 | opaque | <1 μm | isolated |
| 35 000 | opaque | ca. 20 μm | connected channels |

### EXAMPLE 3

### Dependence of the Pore Structure on the Relative PEG Content

The following table shows how the pore structure (determined visually from scanning electron micragraphs and by Hg porosimetry measurements) changes as a function of the relative PEG content (measured in percent by weight) when the molecular weight of PEG is kept constant at 35000

| | PEG (MG 35000) | | | | | |
|---|---|---|---|---|---|---|
| | Weight | % by | Trans- | Pores: SEM | | |
| Sample | [g] | weight | parency | Size | | Type |
| K1 | 0.4 | 2.55 | opaque | 10-20 | μm | isolated |
| K2 | 0.5 | 3.17 | opaque | 30-80 | μm | mainly isolated |
| K3 | 0.6 | 3.78 | opaque | 5-10 | μm | partly connected |
| K4 | 0.7 | 4.38 | opaque | 20-40 | μm | partly connected |
| K5 | 0.8 | 4.98 | opaque | 3-7 | μm | partly connected |
| K6 | 0.9 | 5.56 | opaque | 1-2 | μm | partly connected |
| K7 | 1.0 | 6.14 | opaque | <1 | μm | separated |

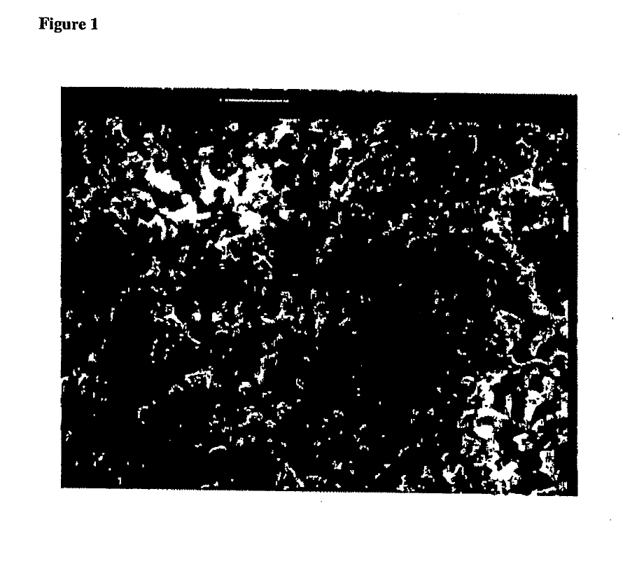

As can be readily seen from the table, the size of the macropores and the degree of connection of the pores to form channels can once again be influenced by varying the relative PEG content. The macropores connected to form channels in the sample K5 are shown in a scanning electron micragraph having a scale of 1 cm≡30 μm in FIG. 1.

### EXAMPLE 4

### Dependence of the Pore Structure on the Relative TEOS Contact

The following table shows how the pore structure (determined visually from scanning electron micragraphs and by Hg porosimetry measurements) changes as a function of the relative TEOS content (measured in percent by weight).

| r value of | | Particle size | Pore Type |
|---|---|---|---|
| TEOS | Transparency | (SEM) | (SEM) |
| 20.57 | opaque | 5-10 μm | particles |
| 17.14 | opaque | 10-20 μm | connected channels |
| 14.7 | opaque | 25-50 μm | connected channels |
| 12.86 | opaque | 10-20 μm | isolated |
| 11.43 | opaque | 30-80 μm | isolated |

### EXAMPLE 5

### Dependence of the Pore Structure on the Concentration of the Solvent in the Solvent Replacement

The concentration of the solvent used in the solvent replacement step (here: ammonium hydroxide replaces nitric acid) is of particular importance in determining the size of the mesopores (measured by means of absorption measurements using nitrogen). Here, the change in significant pore parameters (surface area, pore volume, pore diameter) with changes in the concentration of ammonium hydroxide (1.0 M, 0.1 M, and 0.01 M) is shown at various temperatures.

The assignment of the sample to the respective temperature at concentration of ammonium hydroxide is as shown in the following table

| | | Temperature | | | |
|---|---|---|---|---|---|
| | | 22° C. | 40° C. | 60° C. | 90° C. |
| Concentration | 1.0 M | S1a | S2a | S3a | S4a |
| NH₄OH | 0.1 M | S1b | S2b | S3b | S4b |
| | 0.01 M | S1c | S2c | S3c | S4c |

and the pore parameters for the samples are shown in the table below:

| | | | Pore diameter | |
|---|---|---|---|---|
| | Surface area | Pore volume | [nm] | |
| Sample | [m²/g] | [cm³/g] | Absorption | Desorption |
| S1a | 449.38 | 1.271 | 12.32 | 10.02 |
| S1b | 801.41 | 1.316 | 7.59 | 6.62 |
| S1c | 902.10 | 0.802 | 4.22 | 3.86 |
| S2a | 368.68 | 1.403 | 17.69 | 14.13 |
| S2b | 623.06 | 1.346 | 10.32 | 8.88 |
| S2c | 835.57 | 1.227 | 6.88 | 6.10 |
| S3a | 260.04 | 0.636 | 11.49 | 10.88 |
| S3b | 497.81 | 1.353 | 14.46 | 11.09 |
| S3c | 646.07 | 1.250 | 9.44 | 8.19 |
| S4a | 235.46 | 0.500 | 10.89 | 10.32 |
| S4b | 373.40 | 1.149 | 18.48 | 15.30 |
| S4c | 512.88 | 1.242 | 11.39 | 9.68 |

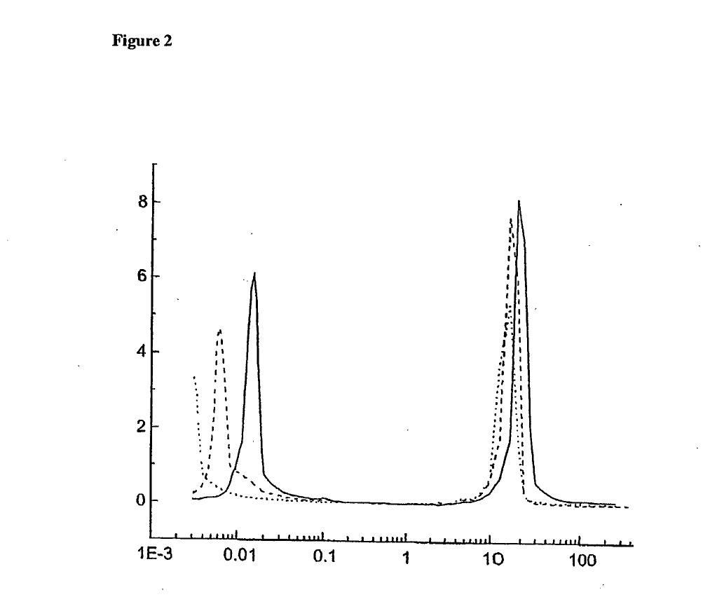

Pore distribution functions for mesopores and macropores of decomposable monoliths produced by the general procedure described in Example 1 and subjected to solvent replacement at 90° C. at various concentrations of ammonium hydroxide are shown in FIG. 2. In this figure, the horizontal x axis gives the mean pore diameter (in microns) and the vertical y axis gives the measured proportion of pores at this diameter (measured in millilitres per gram). The continuous line corresponds to a 2 M ammonium hydroxide solution, the broken line corresponds to a 0.2 M solution and the dotted line corresponds to a 0.02 M ammonium hydroxide solution. The graph thus clearly demonstrates that, in particular, the size of the mesopores can be set reproducibly via the concentration of ammonium hydroxide.

### EXAMPLE 6

### Production of a Decomposable Monolith Having a Trimodal Pore Structure

To obtain the sol, 0.62 g PEG having a molecular weight of 35000 is mixed with 5.5 g of water and 1.3 g of nitric acid. 5.05 g of TEOS are added to this mixture and the mixture is stirred until a clear solution is formed.

At this point, 0.36 g of C₁₄TAB as amphiphile are added to the sol. This means that in this example the PEG content is 4.84% by weight and the C₁₄TAB content is 2.78% by weight. The sol is subsequently poured into moulds, gelled at 40° C. for 5 hours and then aged at 40° C. for 48 hours.

The monolith is then subjected to solvent replacement at 90° C. in 1 M ammonium hydroxide solution for 8 hours. The volume of the solvent is about 10 times the volume of the gel. After solvent replacement is complete, the green body is washed with 0.1 M HNO₃ solution and 25% strength ethanol.

In the drying step, the green bodies are dried at 60° C. for 3 days and subsequently calcined at 500° C. for 5 hours, with the temperature being ramped up at 1 K/min. For monoliths having a trimodal pore structure, calcination is an important step since the C₁₄TAB can be removed quantitatively in this way.

### EXAMPLE 7

### Variation of the Size of the Micropores by Choice of the Amphiphile

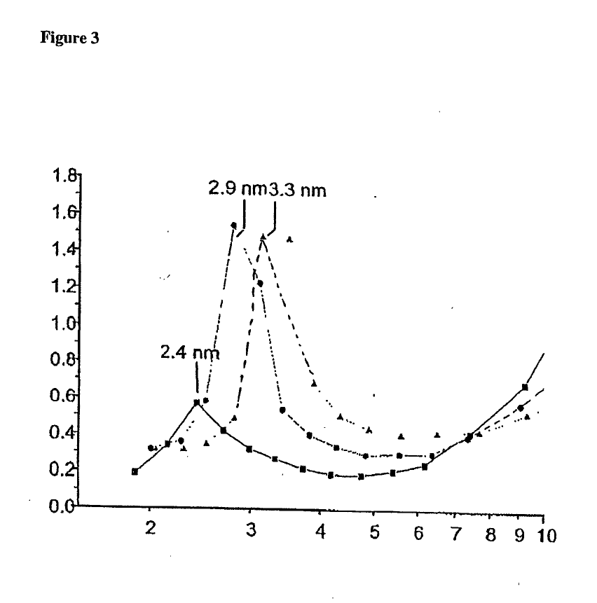

The production of the monolith is carried out exactly as described in example 6, except that C16TAB and C18TAB are now used in place of C14TAB. The influence of the choice of amphiphile on the size of the micropores is shown in FIG. 3. Here, the horizontal x axis gives the pore diameter in nanometers and the vertical y axis gives the pore volume corresponding to the respective pore diameter in cubic centimetres per gram. The diameter of the micropores can clearly be shifted to smaller values by lengthening the aliphatic chain of the amphiphile.

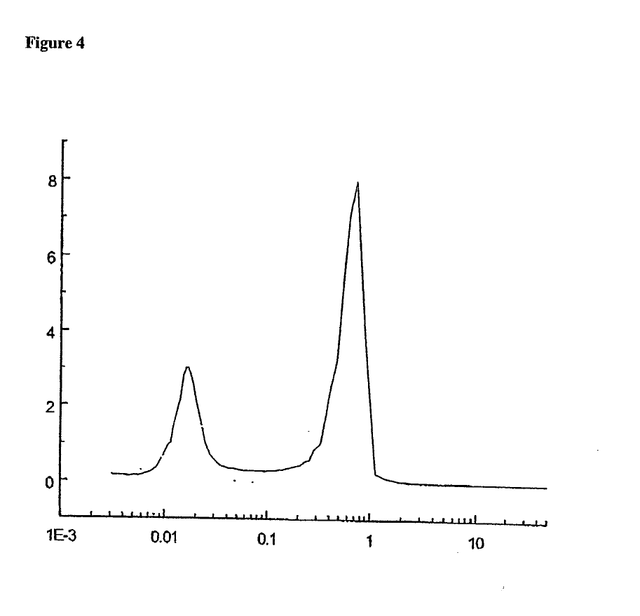

The overall trimodal pore structure of a decomposable monolith obtained with addition of an amphiphile (here C₁₆TAB) is shown by way of example in FIG. 4, where the horizontal x axis gives the pore diameter in microns and the vertical y axis gives the content of the corresponding pores, measured in cubic centimetres per gram. The proportion of micropores and mesopores was determined by means of nitrogen adsorption (dotted line) and the proportion of mesopores and micropores was determined by means of Hg porosimetry (continuous line).

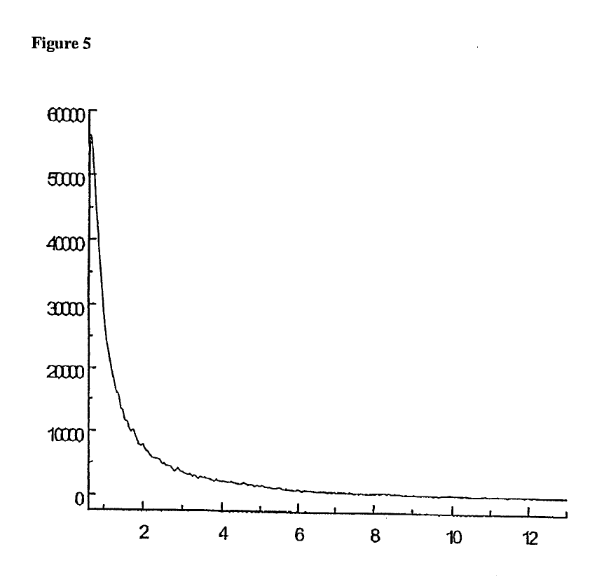

In addition, FIG. 5 shows a small angle X-ray diffraction pattern of the trimodal monolithic material produced using C₁₆TAB The scattering angle to 2Θ in degrees (recorded at the energy of the Cu Kα line) is shown on the horizontal x axis and the relative scattering intensity in arbitrary units is given on the vertical y axis. It can clearly be seen that only the scattered X-rays decreasing from the primary beam (at 0° C.) typical of small angle scattering is observed, but not the Bragg reflections typical of materials containing mesopores (see discussion in the introduction). This demonstrates that the material of the present invention is in fact disordered on all length scales.

### EXAMPLE 8

### Materials According to the Invention Having Active Metal Centres by Cohydrolysis of TEOS with a Metal Alkoxide

The materials according to invention describe in the following example were produced by the method described in Example 1. The following tables gives an overview of the amount of starting materials used:

| | PEG [g] | Water [g] | NH03 35% [g] | TEOS [g} |
|---|---|---|---|---|
| Batch 1 | 1.78 | 15.82 | 3.73 | 14.50 |
| Batch 2 | 1.08 | 16.82 | 3.73 | 14.50 |
| Batch 3 | 0.34 | 15.82 | 3.73 | 14.50 |

The niobium precursor used is a niobium alkoxide (niobium ethoxide). This was mixed with the TEOS and then added to the polymer solution acidified with nitric acid.

The following table gives an overview of the experiments carried out and the properties of the materials after drying and calcination at 400° C.

| | PEG | | | | Stability of | Comment on the | |
|---|---|---|---|---|---|---|---|
| No. | [%] | PEG/Si | H20/Si | Nb/Si | the monolith | structure of the monolith | BET[m²/g] |
| Batch 1 | | | | | | | |
| M24/1 | 4.97 | 0.58 | 14.66 | 0.00 | stable | uniform | 161.9 |
| M24/2 | 4.97 | 0.58 | 14.55 | 0.27 | stable | uniform | 165.1 |
| M24/3 | 4.97 | 0.58 | 14.55 | 0.54 | stable | uniform | 163.3 |
| M24/4 | 4.97 | 0.58 | 14.55 | 2.68 | stable | uniform | 152.9 |
| M24/5 | 4.97 | 0.58 | 14.55 | 5.35 | stable | uniform, no bubbles | 151.2 |
| M24/6 | 4.97 | 0.58 | 14.55 | 26.76 | unstable | very soft and crumbly | |
| Batch 2 | | | | | | | |
| M24/7 | 3.02 | 0.35 | 14.55 | 0.00 | stable | uniform | 185.1 |
| M24.8 | 3.02 | 0.35 | 14.55 | 0.54 | stable | uniform | 178.2 |
| M24/9 | 3.02 | 0.35 | 14.55 | 5.35 | stable | uniform, no bubbles | 163.7 |
| M24/10 | 3.02 | 0.35 | 14.55 | 26.76 | unstable | very soft and crumbly | 128.7 |
| Batch 3 | | | | | | | |
| M24/11 | 0.99 | 0.11 | 14.55 | 0.00 | unstable | very soft and crumbly | 116.5 |
| M24/12 | 0.99 | 0.11 | 14.55 | 0.54 | unstable | very soft and crumbly | 114.7 |
| M24/13 | 0.99 | 0.11 | 14.5 | 5.35 | unstable | very soft and crumbly | 101.4 |
| M24/14 | 0.99 | 0.11 | 14.55 | 26.76 | unstable | very soft and crumbly | 128.2 |

The materials were examined before uniformity of the niobium distribution by means of X-ray fluorescence, giving the following findings: (i) the above of niobium introduced into the synthesis gel were found in the ceramic product and (ii) the niobium introduced is distributed uniformly over the shaped body.

Shaped bodies produced by the above-described method using tantalum ethyoxide, titanium ethyoxide and zirconium ethyoxide in place of the niobium ethyoxide likewise display uniform metal distributions in the siliceous matrix.

### EXAMPLE 9

### Materials According to the Invention Having Active Metal Centres by After-Treatment of the Porous Monolith with a Metal Salt Solution

A monolith produced as described in Example 1 (SiO₂) was activated in a Schlenk flask at 90° C. under an oil pump vacuum for a period of 12 hours. After 12 hours, the flask was cooled to room temperature and flushed with oxygen- and water-free argon. 10 ml of a 0.01 molar solution of vanadium (V) isopropoxide in anhydrous ethanol was introduced through a septum (=bringing into contact). The monolith was heated in the solution at 50° C. for 3 hours, and the solvent was subsequently removed via a Schlenk frit and the monolith was evacuated at 90° C. for 12 hours. This material, too, displays uniform loading with metallic centres and can optionally be calcined.

### EXAMPLE 10

### Use of the Inventive Porous Monolithic Material in the Field of Drug Release

Four different materials have been used, namely (i) as sample Q4, the sample from Example 1 with the difference that the molecular weight of the PEG is 10.000 (diameter of the mesophores: 14 nm), (ii) a sample P4, the sample K5 from example 3 containing an additional 0.5% by weight of C₁₆ TAB (11.3 nm of pore diameter), (iii) as sample P6, sample K5 containing 3.0% by weight of C₁₆ TAB (7.6 nm of pore diameter) and finally (iv) as sample R2, sample K5 from example 3 in which a solvent exchange has been performed in 0.21 m Ammoniumhydroxyde (11 nm of pore diameter).

These samples have been loaded with ibuprofen (diameter of the samples: approximately 0.6 nm) by means of soaking of the monoliths in hexane containing ibuprofen as a desolved drug for three days. Afterwards, the monoliths have been cleaned carefully with hexane and have been dried for one day. The amount of ibuprofene taken up was determined by means of UV-VIS spectroscopy and by means of conventional weighing (see Table).

| | | UV-VIS | | Change of weight | |
|---|---|---|---|---|---|
| | | Adsorbed | | Adsorbed | |
| | | Mass | | Mass | |
| Sample | Weight [mg] | [mg] | Weight % | [mg] | Weights % |
| Q4 | 648.3 | 107.6 | 16.6 | 124.4 | 19.2 |
| P4 | 299.5 | 49.4 | 16.5 | 61.2 | 20.4 |
| P6 | 312.8 | 73.5 | 23.5 | 84.2 | 26.9 |
| R2 | 331.4 | 73.9 | 22.3 | 83.2 | 25.1 |

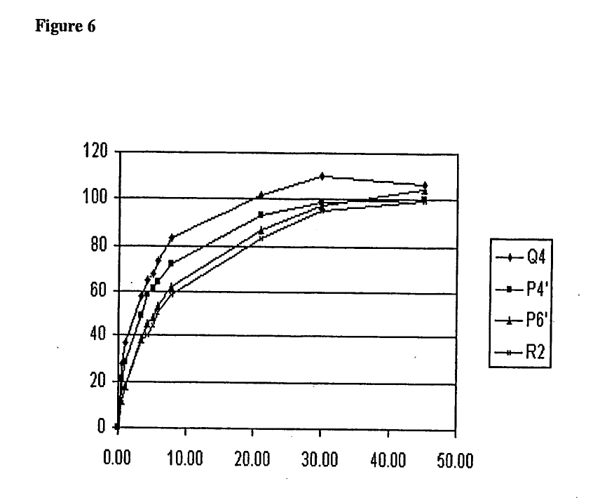

In the above Table, the adsorbed mass in column 3 has been determined by means of UV-VIS spectroscopy while the absorbed mass in column 5 has been determined by measuring the change in weight. In order to investigate the effectiveness of the drug release, the samples have been soaked into a liquid that corresponds to human bodily fluids (at 40° C.) and the concentration of ibuprofen within the liquid has been measured by means of UV-VIS spectroscopy. The amount of ibuprofen set free in the liquid shown in FIG. 6. Here the horizontal axis shows the time in hours and the vertical axis shows the amount of ibuprofen that has been released, given in % with respect to the amount of ibuprofen that had been incorporated into the monoliths at the beginning of the experiment.

As can be seen from FIG. 6, the time at which the drug is released can be determined and/or influenced by the size of the mesophores (as well as by the type of excanging the solvent). As has been expected, the drug release is inhibited for longer times the smaller the pores are.

### EXAMPLE 11

### Loading and Drug Release in the Inventive Monolithic Material that has Not Been Calcined

A decomposable monolith as described in example 6 has been charged with a material, in this case methylene blue, after the drying step at 80° C. (i.e. without a step of calcining) by means of equilibrium absorption. The achieve this, the material has been dipped into a 0.01 m solution of methylene blue in ethanol. The methylene blue is used to model a pharmaceutical drug. After a given period, the monolith has been removed from the solution and cut in half. By means of optical analysis the distribution of the dye over the cross sectional area of the shaped body (monolith) has been measured. This optical analysis shows that after approximately 240 min, an even distribution of the dye over the entire monolith can be found. It is therefore shown that the inventive material can be charged even without having been calcined. The effect of charging is clearly discernable even after having soaked the monolith for about 10 minutens. A gradient between the inner and the outer surface is clearly discernable in this case.

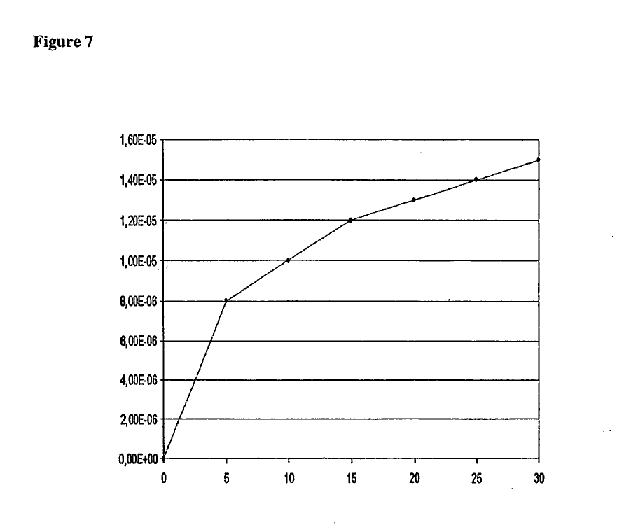

To monitor the “drug” release a monolithic material as described above has been soaked for 24 hours in a dye solution and was then dried and added to a 0.1 m solution of HCl (in order to simulate a physiological solution). FIG. 7 shows the change in dye concentration as measured in the originally pure 0.1 mHCl solution after adding the monolith that has been charged with methylene blue. The Figure shows the dye concentration given in mol/l on the vertical axis as a function of time given in minutes on the horizontal axis.

In summary, it can be stated that this examples shows that (a) the inventive materials can be loaded or charged with a substance even in case the step of calcining is omitted and it can be shown that (b) a release of the model substance (dye) is possible after the charging process under physiological conditions.

1. Monolithic ceramic material having micropores and mesopores or mesopores and micropores or micropores, mesopores and micropores, characterized in that it is at least partially decomposable under physiological conditions.

2. Material according to claim 1, characterized in that structural examination by means of X-ray diffraction displays no Bragg reflections in the small angle region.

3. Material according to claim 1 or 2, characterized in that at least part of the macropores and mesopores or macropores or mesopores present in the material form transport channels running through at least of the material.

4. Material according to at least one of the preceding claims, characterized in that the pore size has at least one property from the following group: the size of the macropores is from 0.1 μm to 100 μm, the size of the mesopores is from 5 nm to 50 nm and the size of the micropores is from 1 nm to 3 nm.

5. Material according to at least one of the preceding claims, characterized in that, after drying, it contains a higher proportion, measured in percent by weight, of inorganic constituents then of organic constituents.

6. Material according to at least one of the preceeding claims, characterized in that it has at least one active metal center integrated into the pore structure.

7. Process for producing a monolithic ceramic material which has micropores and mesopores or mesopores and macropores or micropores, mesopores and micropores and is at least partially decomposable under physiological conditions, characterized in that the process comprises at least the following steps:
(I) bringing a precursor material, a water-soluble polymer, an amphiphilic substance and a hydrolysis catalyst into contact with one another;
(II) inducing the sol-gel transition of the mixture from (I);
(III) at least partially removing and replacing the solvent in the gel from (II) or at least partially removing or replacing the solvent:
(IV) drying the green body obtained from (III).

8. Process according to claim 7, characterized in that an additional calcination step (V)
(V) calcining the dried green body,
is carried out after step (IV).

9. Process according to claims 7 or 8, characterized in that the precursor material used in (I) contains at least one component containing a metal.

10. Process for producing a monolithic ceramic material which has mesopores and macropores and is at least partially decomposable under physiological conditions, characterized in that the process comprises at least the following steps:
(I) bringing a precursor material, a water-soluble polymer and a hydrolysis catalyst into contact with one another;
(II) inducing the sol-gel transition of the mixture from (I);
(III) at least partially removing and replacing the solvent in the gel from (II) or at least partially removing or replacing the solvent:
(IV) drying the green body obtained from (III);
(V) calcining the dried green body at temperatures which do not exceed 500° C. at any point,

11. Process according to claim 10, characterized in that the precursor material used in (I) contains at least one component containing a metal.

12. Process according to at least one of claims 7 to 11, characterized in that the precursor material is selected from the group consisting of completely hydrolysable alkoxides, alkoxides having at least one group which cannot be hydrolysed; halides which are decomposable in aqueous solution, polymerizable metal salts, oligomeric precursor materials, organically modified silicates; coordination compounds having carboxyl or β-diceton ligands; and combinations of two or more of the abovementioned substances.

13. Process according to at least one of claims 9 or 11, characterized in that the metal-containing component(s) of the precursor material is/are selected from the group consisting of organometallic compounds, metallocenes, metallic colloid, metal alkoxides and combinations of two or more of the abovementioned substances.

14. Process according to at least one of claims 7 to 13, characterized in that the hydrolysis catalyst is selected from the group consisting of basic substances such as ammonium, amines, ammonium ions solutions; acidic substances such as mineral acids, organic acids, fluoride-containing solutions; and combinations of two or more of the abovementioned substances.

15. Process according to at least one of claims 1 to 14, characterized in that the water-soluble polymer is selected from the group consisting of uncharged and ionic polymers, and combinations and mixtures of two or more of the abovementioned substances.

16. Process according to at least one of claims 1 to 15, characterized in that TEOS or a metal alkoxide or TEOS and a metal alkoxide is used as precursor material, nitric acid is used as hydrolysis catalyst, PEG is used as water-soluble polymer and CTAB is used as optional amphiphilic substance, and in that the PEG content is in the range from 2 to 10% by weight, based on the total weight, in that the molecular weight of the PEG is from 10000 to 50000, in that the relative proportion of TEOS, given as the “r” value, is in the range from 10 to 20, in that the proportion of CTAB which is used optionally is from 0.01 to 5% by weight, based on the total weight, and in that the solvent for the solvent replacement is ammonium hydroxide.

17. Process according to at least one of claims 1 to 16, characterized in that the material of the invention is subjected to an after-treatment which may be selected from the group consisting of: impregnating the monolithic ceramic material with, or bringing it into contact with, catalytically active substances, organic compounds or mixtures or further auxiliaries or additives, functionalising at least parts of the internal surface, lipophilising the material; successively loading the pores and all combinations of two or of the above steps.

18. Process according to claim 17, characterized in that the substance or substances for the impregnation or contacting comprises at least one carbon-containing precursor compound which contains at least one carbon atom.

19. Process according to claim 17 or 18, characterized in that the carbon-containing material of the invention is calcined at temperatures above 500° C. under nitrogen after impregnation or contacting and is subsequently carbonized under reduced pressure.

20. Process according to at least one of claims 17 to 19, characterized in that the material of the invention in calcined after the after-treatment.

21. Material obtainable by a process according to any of claims 7 to 20.

22. Use of the monolithic ceramic material according to at least one of claims 1 to 6 or the monolithic ceramic material obtainable by a process according to at least one of claims 7 to 20 as all-active catalyst, as catalyst support, as molecular sieve, as biological separator, having a sharp cut off criteria in respect of the molecular weight, as osmotic membrane or as dielectric medium.

23. Use of the monolithic ceramic material according to at least one of claims 1 to 6 or the monolithic ceramic material obtainable by a process according to at least one of claims 7 to 20 for the temporally delayed and temporally controlled and regionally defined supply of substances of dyes, cosmetic active compounds, auxiliaries or additives, pharmaceutical relative substances, proteins, peptides, enzymes, active compounds derived from plants, nutrients or nutrient additives, animal feeds or animal feed additives, fragrance or flavours.

24. Use of the monolithic ceramic material according to at least one of claims 1 to 6 or the monolithic ceramic material obtainable by a process according to at least one of claims 7 to 20 as biodegradable or biologically resorbable or as biologically integrable ceramic material in medical technology, in particular for strengthening bone, for supporting connective tissue and for the healing of wounds.

25. Use of the monolithic carbon-containing material obtainable by a process according to at least one of claims 17 to 20 as storage material for hydrogen or as support material for a hydrogen storage material.

26. Use of the monolithic carbon-containing material obtainable by a process according to at least one of the claims 17 to 20 in combination with or doped with at least one medium capable of reversibly storing hydrogen selected from the group consisting of hydrides of the main group and transition metals, semimetal hydrides, mixed hydrides, alanates and mixtures of at least two of this substances.
